(19) United States
(12) Patent Application Publication
(10) Pub. No.: US 2005/0261645 A1
(43) Pub. Date: Nov. 24, 2005

(54) Ostomy appliance

(76) Inventors: Wayne Conrad, Hampton (CA); Nina Conrad, Hampton (CA); Brock Masters, Hampton (CA)

Correspondence Address: BERESKIN AND PARR, 40 KING STREET WEST, BOX 401, TORONTO, ON, M5H 3Y2, CA

(21) Appl. No.: 10/958,650

(22) Filed: Oct. 6, 2004

(30) Foreign Application Priority Data
May 18, 2004 (CA) 2,467,413

(51) Int. Cl. A61F 5/44

(52) U.S. Cl. 604/332

(57) ABSTRACT

An ostomy pouch is constructed in a manner to disguise its actual function by use of colors, surface textures, iconic perimeter shapes and/or iconic shapes applied to the surface of the ostomy pouch. The ostomy pouch may be made of a blue, red, black, yellow, orange, purple or bright pink plastic material as supposed to a flesh tone of thick current ostomy pouches are made. The surface of the plastic of which the ostomy pouch is constructed is preferably textured to give it the visual appearance of cloth such as linen, cotton, burlap, or canvas. The pouch is constructed to look like a small purse, a heart, a butterfly, a seashell, a teddy bear head, a teddy bear, a dog, a cat, or another recognizable icon to allow the pouch to become a decorative feature rather than a medical appliance to be hidden.

### FIELD OF THE INVENTION

The present invention relates to ostomy appliances and in particular a pouch that is used for the collection of the waste material.

### BACKGROUND OF THE INVENTION

A person whose normal bowel function or bladder is impaired due to birth defect, injury, disease, or other disorder may undergo surgery known as an “ostomy.” Ostomy surgery often involves removal or bypass of impaired or diseased tissue and the creation of a hole in the abdominal wall of the patient, and pulling a section of the bladder conduit or bowel to the skin surface and subsequently attaching it to the skin surface. Bowel attachment surgery is termed an “ileostomy” when the small intestine is involved, termed a colostomy when the large intestine is involved, and termed a urostomy when the bladder is involved.

The hole in the skin to which the end of the bladder conduit or intestine is attached is called the “stoma,” and the surrounding skin area is called the “peristomal area”. The ileostomy and colostomy stoma provides an open conduit through which a constant or intermittent efflux of fecal material can occur and the urostomy stoma allows the efflux of urine. The urine or feces is collected in a bag-like ostomy appliance which is worn by the user and replaced by the user as required every few days. In this application, the word ostomy is used to refer to an ileostomy, colostomy, urostomy or any like procedures, which now exist or which may be developed in the future and which result in the efflux of urine and/or fecal matter through a surgically created opening in the skin of a person.

The attachment of the ostomy appliance to the user can be achieved with either a one piece or a two-piece system. A two-piece system typically consists of a flange which is adhered to the body of the person with the ostomy, and a bag like device to provide collection of the feces or urine. The flange and the bag each provide a mechanical member which allows the bag to be removably fastened to the flange and for a plurality of bags to be used with a single flange over a period of 2-7 days. The one-piece system integrates both the means of adhering to the body and the bag collection device. A modern adhesive system for ostomy appliances typically consists of an inner hydrocoloid layer over which a protective paper or cloth tape is adhered to provide a barrier through which feces and urine will not pass but which does allow oxygen and water vapor to pass.

Typically, current ostomy appliances are typically made of a smooth “pink skin color” plastic meant to simulate Caucasian flesh tone. In particular, current ostomy appliances are typically polyvinyl chloride (PVC) film, which may optionally be covered by a woven cotton layer. The visual appearance of the devices is quite “clinical”. The use of pink skin color plastic of current designs does not blend into the skin and is therefore an unsuccessful attempt to hide the ostomy pouch by blending into the skin.

### SUMMARY OF THE INVENTION

This invention relates to the change of the visual appearance of the ostomy pouch in terms of color, surface texture, and overall shape to visually make it appear like a fashion accessory or other common object thereby reducing the stress of the user who might otherwise be embarrassed by the appearance of the medical appliance. The invention is applicable to both one piece and two-piece appliances.

Accordingly, this invention relates to the configuration and/or surface decoration of an ostomy appliance so as to render the ostomy appliance more acceptable to a user and/or to make the ostomy appliance part of an outfit. According to this invention, the ostomy pouch is provided with at least one decorative feature including, but not limited to, attributes of texture, color, shape and surface decoration. In order to the ostomy appliance more acceptable to children who must use an ostomy appliance, the appliance may be configured as a familiar shape such as a flower, an animal, a heart or other fanciful shape. In order to the ostomy appliance more acceptable to adults who must use an ostomy appliance, the appliance may be designed to appear to be part of an outfit such as a waist pouch, a part of a belt, a purse or the like.

Accordingly, in accordance with one aspect of the invention, there is provided an ostomy appliance wherein the ostomy appliance has the appearance that disguises the identity of the ostomy appliance.

In one embodiment, the ostomy appliance is provided with at least one attribute of texture, color, shape and surface decoration to disguise the identity of the ostomy appliance.

In another embodiment, the ostomy appliance has the appearance of a fashion accessory.

In another embodiment, the ostomy appliance has the appearance of part of an outfit.

In another embodiment, the ostomy appliance has the appearance of an design that is pleasing to a child.

In another embodiment, the ostomy appliance has the appearance of an animal, a caricature of an animal, a portion of an animal, a caricature of part of an animal, an insect, a caricature of an insect, a portion of an insect, a caricature of part of an insect, a sea shell, a heart or a flower.

In another embodiment, the ostomy appliance has the appearance of a teddy bear.

In another embodiment, the ostomy appliance has a front surface that is at least substantially opaque.

In another embodiment, the ostomy appliance has a front surface that is essentially opaque.

In another embodiment, the ostomy appliance has a front surface that is coloured sufficiently such that the contents of the pouch are not viewable.

In another embodiment, the ostomy appliance has a front surface that is black, brown, red, orange, yellow, green, blue, violet, gray, and/or white.

In another embodiment, the ostomy appliance has a front surface that is textured.

In another embodiment, the front surface is patterned with dimples which are 0.001″ to 0.100″ in diameter with a depth 1-75% of the diameter of the dimple.

In another embodiment, the front surface is patterned with dimples which are 0.003 to 0.050″″ in diameter with a depth 5-35% of the diameter of the dimple.

In another embodiment, the front surface is patterned with dimples which are 0.005 to 0.015″ in diameter with a depth 15%-20% of the diameter of the dimple.

In another embodiment, the ostomy appliance has a front surface that has fibers provided thereon.

In another embodiment, the fibers are 0.001 to 0.125″ long with a diameter that is between 1% to 1000% of the length of the fibers.

In another embodiment, the fibers are 0.003 to 0.040″ long with a diameter that is between 5% to 300% of the length of the fibers.

In another embodiment, the fibers are 0.005″ to 0.015″ long with a diameter that is between 35% to 100% of the length of the fibers.

### BRIEF DESCRIPTION OF THE DRAWINGS

These and other advantages of the instant invention will be better understood from the following description of the preferred embodiments and the accompanying drawings in which:

FIG. 1 is a front elevation view of an ostomy appliance according to a preferred embodiment of the instant invention wherein the ostomy pouch 1 forms a recognizable shape, in this case, the shape of a teddy bear;

FIG. 2 is an exploded view of the assembly of FIG. 1;

FIG. 3 is a perspective view of the ostomy appliance of FIG. 1;

FIG. 4 is a rear elevation view of an alternate embodiment of the ostomy appliance of FIG. 1 wherein the ostomy appliance is one-piece version of the ostomy appliance of FIG. 1;

FIG. 5 is a front elevation view of a further alternate embodiment of wherein the ostomy pouch 12 is in the shape of a heart;

FIG. 6 is a front elevation view of a further alternate embodiment wherein the ostomy pouch 15 is in the shape of a seashell;

FIG. 7 is a front elevation view of a further alternate embodiment wherein the ostomy pouch 20 is in the shape of a flower; and,

FIG. 8 is a front elevation view of a further alternate embodiment wherein the ostomy pouch 23 is in the shape of the head of a dog.

### DETAILED DESCRIPTION OF THE INVENTION

The following description describes embodiments for disposable colostomy pouches. It will be appreciated by those skilled in the art that the invention may be utilized with ostomy appliances of all constructions. For example, it will be appreciated that the invention described herein is equally applicable to all ostomy pouches including urostomy and ileostomy pouches. Further, while the drawings show the invention as it relates to filtered pouch, the invention is equally applicable to filter-less pouches or vented pouches.

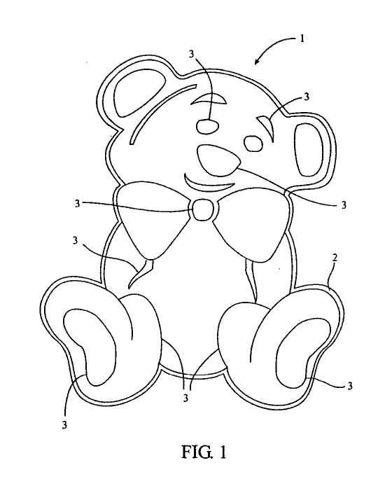

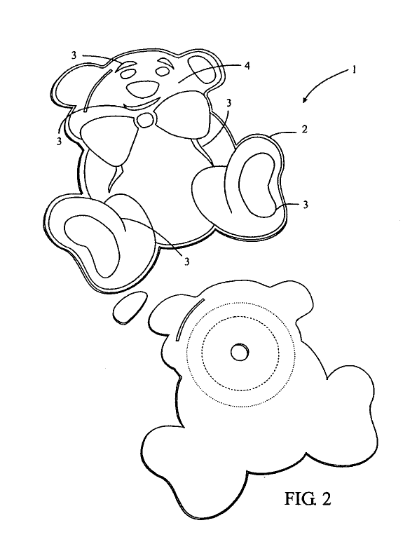

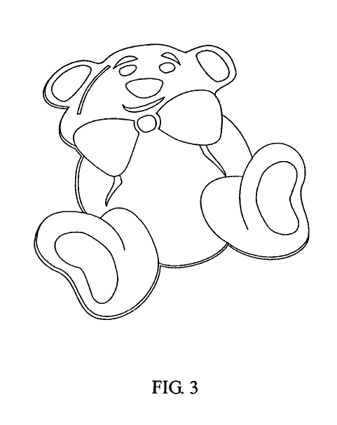

An ostomy appliance comprises a pouch 1 and means to secure pouch 1 to a user. As shown in FIGS. 1-3, ostomy pouch 1 forms a recognizable shape, in this case, the shape of a teddy bear. Ostomy pouch 1 may be constructed by any technique known in the art. For example, ostomy pouch 1 may be formed by heat-sealing a front plastic sheet or layer 4 to a rear plastic sheet or layer 5 along their perimeter 2. Layers 4 and 5 may be made of any material known in the ostomy art. Preferably, pouch 1 is constructed of two or more layers of thin film heat sealable plastic, preferably PVC, and preferably the plastic is 0.001″ to 0.020″ thick, more preferably 0.002 to 0.010″ thick and most preferably 0.004 to 0.007″ thick.

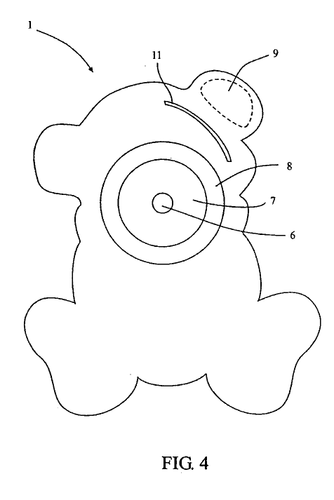

The osotmy appliance may be secured to a user by any means known in the art. For example, the adhesive for a one-piece system or the ring for a two-piece system may be affixed to rear layer 5 of ostomy pouch 1. For example, FIG. 4 shows a one-piece version of the ostomy pouch 1 of the instant invention. Rear sheet 5 has a hole 6 for the stoma to penetrate, a hydrocolic adhesive layer 7 surrounding hole 6 and an outer paper or urethane tape adhesive layer 8 which covers the hydrocolic adhesive layer 7 until pouch 1 is ready for use. A filter 9 may be provided and may be located at any location in the ostomy appliance that is known in the art. Preferably, filter 9 is located at an upper portion of pouch 1 and, more preferably, is provided in a pocket that is created by a heat seal 11 to protect the filter from direct contact with fecal matter. The configuration of pouch 1 may be utilized to create a suitable pocket. For example, as shown in FIG. 4, filter 9 is provided in ear 10 of the teddy bear. Heat seal 11 is positioned to generally close ear 10 so as to create a pocket for filter 9.

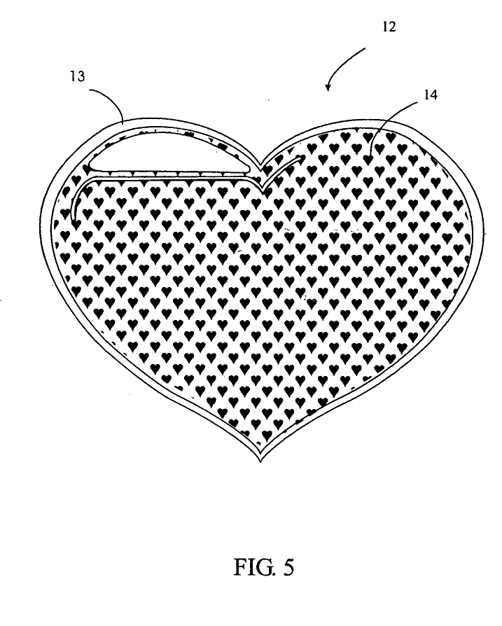

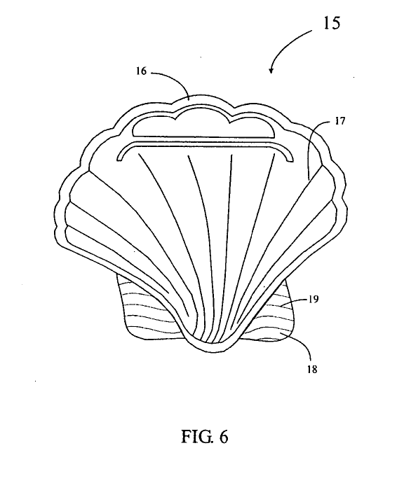

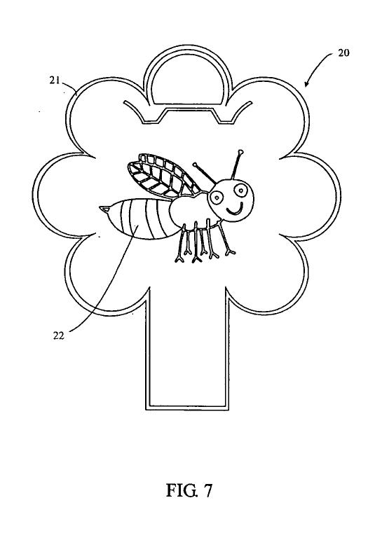

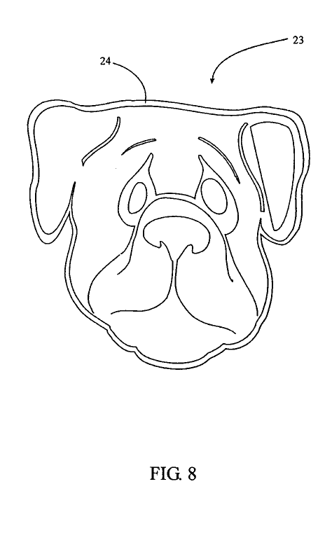

The shape of the ostomy pouch of the instant invention camouflages the true nature of the ostomy pouch 1 due to its recognizable iconic shape. For example, pouch 1 may be in the shape of a teddy bear, a heart (see FIG. 5), a seashell (see FIG. 6), a flower (see FIG. 7), the head of a dog (see FIG. 8) a dog, a butterfly or an insect or caricatures thereof. Accordingly, when pouch 1 is viewed, it may appear to an observer as a fanciful waist pouch for a child having a particular shape. This appearance may be heightened if the pouch is coloured sufficiently such that the contents of the pouch are not viewable. Accordingly, preferably layer 4, and more preferably layers 4 and 5, are at least substantially opaque and, more preferably, opaque.

As shown in FIG. 1, lines 3 that form the patterns of the features of the bear may be defined by the front and rear layers 4 and 5 being heat sealed together. Alternately the design may be printed, e.g., silk screened, on front layer 4. It will be appreciated that front layer 4 may be larger than the design. Preferably, pouch 1 has the same shape as the design. It will be appreciated that the design may be created by any combination of the shape of layers 4 and 5 and printing. For example, the perimeter lines 3 may be prepared by front and rear layers 4 and 5 being heat sealed together and interior lines 3 may be printed.

For several reasons, a child may have to have an ostomy. An advantage of the present invention is that by forming the ostomy appliance in a shape that is familiar to a child, the child will be more accepting of wearing the appliance and possibly more self confident. Current pouches are utilitarian and have a clinical appearance. Ostomy appliances therefore appear to a child as a medical device. While this is the case, it is important for a child to feel normal. According to the instant invention, an ostomy appliance may be in any shape that is pleasing to a child, or an adult.

The color of the plastic of which this ostomy pouch 1 is constructed is preferably black, brown, red, orange, yellow, green, blue, violet, gray, and/or white. Preferably, a sufficient amount of colour is provided such that the pouch is substantially opaque and, more preferably, opaque.

The surface of the plastic of which the pouch is constructed may be smooth but is preferably textured, such as by being patterned with dimples which are 0.001″ to 0.100″ in diameter, more preferably 0.003 to 0.050″ in diameter and most preferably 0.005 to 0.015″ in diameter with a depth 1-75% of the diameter of the dimple, more preferably having a depth 5-35% of the diameter of the dimple and most preferably 15%-20% the diameter of the dimple. This dimpling or texturing pattern serves to alter the appearance of the plastic by forming a mechanical pattern similar to the appearance and texture of fabrics. Accordingly, texturing of the plastic can be used by itself or with other attributes provided herein to disguise the function of the ostomy appliance to a casual observer.

As shown in FIG. 5, ostomy pouch 12 is in the shape of a heart that is heat sealed along the perimeter 13. In this embodiment, the front surface, and optionally the front and side surfaces of ostomy pouch 12 is covered with a layer of short fibers 14. Fibers 14 may be adhered to the plastic of which the ostomy appliance is constructed by any means known in the art, such as an adhesive that is not readily broken down by contact with water or body fluids. The short fibers are preferably 0.001 to 0.125″ long, more preferably 0.003 to 0.040″ long and most preferably 0.005″ to 0.015″ long having a diameter between 1% to 1000% of their length, more preferably 5% to 300% of their length and most preferably a diameter between 35% to 100% of their length.

The layer of short fibers 14 may be applied to an ostomy appliance to create a design or to enhance a design. Fibers 14 may be applied by any means known in the art. For example, fibers 14 may be applied by electrostatic flocking.

Fibers 14 may be applied to create a desired fiber density and/or pattern. Fibers 14 may be applied over the entire surface of a pouch. For example, the pouch may be made from a black plastic and the entire surface that is visible when the ostomy appliance is worn by a user may be covered in black fibers. Such a design would give the pouch the appearance of being an accessory for a belt. The colour and pattern may be varied depending upon the clothing of the user. The pouch may be checkered, stripped, an abstract design or a still life pattern or the like.

Alternately, fibers 14 may be applied in a pattern such that the short fibers form a decorative pattern on the surface of the pouch. For example, as shown in FIG. 5, fibers 14 are applied in a heart pattern. Optionally, a mixture of different colors of flock may be applied together to form an appearance similar to cloth and other textiles. Optionally, different colors of short fibers may be applied to different regions of the plastic of which the pouch is constructed to form decorative or iconic patterns. The plastic of the front layer may have a complementary or contrasting colour.

It will be appreciated that if the fibers have a sufficient density to prevent light to pass therethrough that the plastic from which the pouch is made need not be coloured and need not be opaque. The layer of fibers will prevent a third party from viewing the contents of the ostomy appliance.

The attributes may be combined in any desired combination. For example, as shown in FIG. 6, the layers 4 and 5 of ostomy pouch 15 are in the shape of a seashell which is heat-sealed along the perimeter 16. Portions 17 of the surface of this embodiment are silk screened to create the lines of the shell and the base of the shell 18 is covered with a layer of short fibers 19.

As shown in FIG. 7, the layers 4 and 5 of ostomy pouch 20 are in the shape of a flower which is heat sealed along the perimeter 21. A small bee character 22 is applied to the surface of the pouch by means of silk screening. Optionally, this decoration may be applied by heat lamination or by flocking short fibers onto the pouch surface in the pattern of the bee character 22. As shown in FIG. 8 the layers 4 and 5 of ostomy pouch 23 are in the head of a dog which is heat-sealed along the perimeter 24.

1. An ostomy appliance wherein the ostomy appliance has the appearance that disguises the identity of the ostomy appliance.

2. The ostomy appliance of claim 1 wherein the ostomy appliance is provided with at least one attribute of texture, color, shape and surface decoration to disguise the identity of the ostomy appliance.

3. The ostomy appliance of claim 1 wherein the ostomy appliance has the appearance of a fashion accessory.

4. The ostomy appliance of claim 1 wherein the ostomy appliance has the appearance of part of an outfit.

5. The ostomy appliance of claim 1 wherein the ostomy appliance has the appearance of an design that is pleasing to a child.

6. The ostomy appliance of claim 5 wherein the ostomy appliance has the appearance of an animal, a caricature of an animal, a portion of an animal, a caricature of part of an animal, an insect, a caricature of an insect, a portion of an insect, a caricature of part of an insect, a sea shell, a heart or a flower.

7. The ostomy appliance of claim 7 wherein the ostomy appliance has the appearance of a teddy bear.

8. The ostomy appliance of claim 1 wherein the ostomy appliance has a front surface that is at least substantially opaque.

9. The ostomy appliance of claim 1 wherein the ostomy appliance has a front surface that is essentially opaque.

10. The ostomy appliance of claim 1 wherein the ostomy appliance has a front surface that is coloured sufficiently such that the contents of the pouch are not viewable.

11. The ostomy appliance of claim 1 wherein the ostomy appliance has a front surface that is black, brown, red, orange, yellow, green, blue, violet, gray, and/or white.

12. The ostomy appliance of claim 1 wherein the ostomy appliance has a front surface that is textured.

13. The ostomy appliance of claim 12 wherein the front surface is patterned with dimples which are 0.001″ to 0.100″ in diameter with a depth 1-75% of the diameter of the dimple.

14. The ostomy appliance of claim 12 wherein the front surface is patterned with dimples which are 0.003 to 0.050″″ in diameter with a depth 5-35% of the diameter of the dimple.

15. The ostomy appliance of claim 12 wherein the front surface is patterned with dimples which are 0.005 to 0.015″ in diameter with a depth 15%-20% of the diameter of the dimple.

16. The ostomy appliance of claim 1 wherein the ostomy appliance has a front surface that has fibers provided thereon.

17. The ostomy appliance of claim 16 wherein the fibers are 0.001 to 0.125″ long with a diameter that is between 1% to 1000% of the length of the fibers.

18. The ostomy appliance of claim 16 wherein the fibers are 0.003 to 0.040″ long with a diameter that is between 5% to 300% of the length of the fibers.

19. The ostomy appliance of claim 16 wherein the fibers are 0.005″ to 0.015″ long with a diameter that is between 35% to 100% of the length of the fibers.
